(19) United States
(12) Patent Application Publication
(10) Pub. No.: US 2013/0196093 A1
(43) Pub. Date: Aug. 1, 2013

(54) SYSTEMS AND METHODS FOR DIFFUSING ESSENTIAL OILS

(71) Applicant: Jonathan Richard Fairbanks, Orem, UT (US)

(72) Inventor: Jonathan Richard Fairbanks, Orem, UT (US)

(21) Appl. No.: 13/746,735

(22) Filed: Jan. 22, 2013

Related U.S. Application Data
(60) Provisional application No. 61/589,232, filed on Jan. 20, 2012.

(51) Int. Cl. A61L 9/14; B65D 90/00

(52) U.S. Cl. CPC A61L 9/14; B65D 90/00
USPC 428/34.1; 512/5; 222/251; 222/394; 206/459.1

(57) ABSTRACT

The present invention relates to essential oils. In particular, some implementations of the present invention relate to systems and methods for diluting and diffusing one or more essential oils from a portable dispenser. In some cases, the described systems include a solution comprising one or more essential oils, emulsifiers, and diluents, wherein the solution is disposed in a container that is capable of diffusing the solution. While the various ingredients can be present at any suitable concentration, in some instances, a ratio between the volume of the essential oil and the volume of the emulsifier is between about 2:1 and about 1:2. In some instances, a ratio of the volume of the essential oil to the volume of the diluent is between about 1:4 and about 1:8. Other implementations are also disclosed.

### CROSS-REFERENCE TO RELATED APPLICATION

This application claims priority to U.S. Provisional Patent Application Ser. No. 61/589,232, filed Jan. 20, 2012, and entitled “SYSTEMS AND METHODS FOR DIFFUSING ESSENTIAL OILS;” the entire disclosure of which is hereby incorporated by reference.

### BACKGROUND OF THE INVENTION

1. Field of the Invention

The present invention relates to essential oils. In particular, some implementations of the present invention relate to systems and methods for diluting one or more essential oils and diffusing such oils from a portable dispenser.

2. Background and Related Art

Generally speaking, essential oils are concentrated hydrophobic liquids containing volatile aromatic chemicals that have been taken from one or more plants through a process involving an extraction technique, such as distillation, expression, and/or solvent extraction. In this regard, the oils are considered “essential” because they can carry a distinctive scent or the “essence” of the plant.

Essential oils are often used for a wide variety of purposes. For instance, essential oils can be used in the production of perfumes, cosmetics, and soaps; as flavoring agents for foods or drinks; in or as medicaments (e.g., as a treatment for skin, cancer, etc.; to provide aromatherapy; etc.); and for a variety of other purposes.

While essential oils have been found to have a variety of desirable uses, some methods for dispensing essential oils have shortcomings. In some cases, because essential oils are hydrophobic, some essential oils are diluted in a carrier oil. In such cases, the essential oils can leave an undesirable oily residue when they are applied to a surface, such as skin. In some other cases, oils are provided without dilution. In such cases, however, it may be easy to dispense more oil (which may be relatively expensive) than is necessary to obtain a desired effect. In still other cases, where essential oils are heated or burned to diffuse a desired scent, such methods may be somewhat dangerous and/or be of limited application. In yet other cases, in order to dilute and diffuse essential oils for aromatic benefits, one must use a conventional electric or battery-powered diffuser. In such cases, however, the diffuser may: only be used where it can be plugged in; be bulky; be subject to battery drain; require pure, undiluted oils; and/or be relatively expensive to use.

Thus, while techniques currently exist that are used to dispense essential oils, challenges still exist. Accordingly, it would be an improvement in the art to augment or even replace current techniques with other techniques.

### SUMMARY OF THE INVENTION

The present invention relates to essential oils. In particular, some implementations of the present invention relate to systems and methods for diluting one or more essential oils and diffusing such oils from a portable dispenser.

Some implementations of the present invention take place in association with one or more essential oils that are emulsified and diluted in a container. In this regard, the essential oil can comprise any suitable essential oil that can be mixed with an emulsifier and be diluted in water (and/or any other suitable diluent). Some non-limiting examples of essential oils include oil extracts of sandalwood, oranges, roses, mentha avensis, peppermint, cedarwood, lemons, eucalyptus globulus, litsea cubeba, clove, spearmint, orange, lavender, any other suitable plant, and combinations thereof.

With respect to the emulsifier, the emulsifier can comprise any suitable chemical that is capable of emulsifying one or more essentials oils so that the oils are miscible with, and can be diluted in, water (or another aqueous diluent). In this regard, some examples of suitable emulsifiers include, but are not limited to, polysorbate (e.g., polysorbate 20, polysorbate 40, polysorbate 60, polysorbate 80, etc.), sodium stearoyl lactylate, ceteareth 20, diacetyle tartaric (acid) ester of monoglyceride, lecithin, cetearyl alcohol, and/or any other suitable emulsifying agents

The emulsifier can be mixed with the essential oil at any suitable ratio that allows the essential oil to be emulsified and diluted in a diluent, such as water (e.g., distilled water). For instance, the emulsifying agent can be mixed with the total amount of essential oil at a ratio that is as high as a ratio selected from about 2:1 (emulsifying agent to essential oil), about 3:1, about 5:1, and about 10:1; or that is as low as a ratio selected from about 1:10, about 1:5, about 1:2, and about 1:1. Furthermore, the emulsifying agent can be added to the essential oil at any suitable combination or sub-range of the aforementioned ratios (e.g., between about 2:1 and about 1:2 (e.g., about 1:1)).

The essential oil and emulsifier can be diluted in any suitable diluent or solvent, including, without limitation, water and/or another aqueous solvent. In this regard, the essential oil and emulsifier can be diluted in any suitable amount of the diluent that allows the emulsified essential oil to be at least partially dissolved in the diluent. In one example, the ratio of diluent to essential oil (or the emulsifier) is as high as a ratio selected from about 200:1, about 100:1, about 20:1, about 10:1, and about 7:1. In another example, the ratio of the diluent to the essential oil (or the emulsifier) is as low as a ratio selected from about 1:1, about 2:1, about 3:1, about 4:1, and about 5:1. In still another example, the ratio of the diluent to essential oil (or the emulsifier) is found in any suitable combination or sub-range of the aforementioned ratios (e.g., between about 5:1 and about 7:1, or about 6:1).

With respect to the container, the container can comprise any suitable vessel that is capable of holding the essential oil, emulsifier, and diluent. By way of example, the container can comprise a spray bottle, a vial, a jug, a canister, and/or any other suitable object. In some instances, however, the container comprises a vessel that is capable of spraying or otherwise diffusing the emulsified and diluted essential oil. Indeed, in some embodiments, the container comprises a positive displacement pump, a negative displacement pump, an aspirator nozzle, an atomizer nozzle, an aerosol canister, a pressurized vessel, a motorized pump, and/or any other mechanism that is capable of spraying, misting, expelling, or otherwise diffusing the emulsified and diluted essential oil. Indeed, in some instances, the container comprises a portable spray bottle that is capable of spraying a fine mist of the essential oil emulsification (or the emulsified and diluted oil).

While the methods and processes of the present invention have proven to be particularly useful in the area of spraying emulsified and diluted essential oil to diffuse a scent of the essential oil (e.g., as an air freshener), those skilled in the art can appreciate that the described methods and processes can be used in a variety of different applications and in a variety of different areas of manufacture. For instance, the skilled artisan will recognize that the described systems and methods can be used for dispensing one or more essential oils for therapeutic reasons, medicinal reasons, for flavoring food or drinks, and/or for virtually any other purpose in which the use of an emulsified and diluted essential oil may be beneficial.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE FIGURE

In order that the manner in which the above recited and other features and advantages of the present invention are obtained, a more particular description of the invention will be rendered by reference to specific embodiments thereof, which are illustrated in the appended Figures. Understanding that the Figures depict only typical embodiments of the present invention and is not, therefore, to be considered as limiting the scope of the invention, the present invention will be described and explained with additional specificity and detail through the use of the accompanying Figures in which:

FIG. 1 illustrates plan view a representative system for dispensing one or more emulsified and diluted essential oils;

FIG. 2 illustrates an exploded, perspective view of a representative embodiment of a container for diffusing one or more emulsified and diluted essential oils; and

FIG. 3 illustrates a plan view of a representative embodiment of the container.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of suitable essential oils, emulsifiers, diluents, containers, etc., to provide a thorough understanding of embodiments of the invention. One having ordinary skill in the relevant art will recognize, however, that the invention may be practiced without one or more of the specific details, or with other methods, components, materials, and so forth. In other instances, well-known structures, materials, and/or operations are not shown or described in detail to avoid obscuring aspects of the invention.

The present invention relates to essential oils. In particular, some embodiments of the present invention relate to systems and methods for diluting one or more essential oils and diffusing such oils from a portable dispenser. While the described systems and methods can comprise any suitable ingredient and/or component that allow an emulsified and diluted essential oil (or an essential oil emulsification) to be dispensed, some embodiments of the present invention include one or more essential oils, emulsifiers, diluents, and containers.

With regard to the essential oil, the essential oil can comprise virtually any suitable essential oil that can be mixed with an emulsifier (discussed below) and that can be diluted in a diluent (discussed below). Some examples of suitable essential oils include, but are not limited to, agar oil, ajwain oil, aloe vera oil, Angelica root oil, anise oil, asafetida oil, balsam oil, basil oil, black pepper oil, bergamot oil, buchu oil, birch oil, camphor, cannabis flower essential oil, caraway oil, cardamom seed oil, carrot seed oil, chamomile oil, calamus root oil, cinnamon oil, cistus oil, citronella oil, clary sage oil, clove leaf oil, coffee oil, coriander oil, costmary oil, costus root oil, cranberry seed oil, cubeb oil, cumin oil, black seed oil, cypress oil, cypriol oil, curry leaf oil, davana oil, dill oil, elecampane, eucalyptus oil, fennel seed oil, fenugreek oil, fir oil, frankincense oil, galangal oil, galbanum oil, geranium oil, ginger oil, goldenrod oil, grapefruit oil, henna oil, helichrysum oil, horseradish oil, hyssop oil, Idaho tansy oil, jasmine oil, juniper berry oil, laurus nobilis oil, lavender oil, ledum oil, lemon oil, lemongrass oil, lime oil, litsea cubeba oil, mandarin oil, marjoram oil, melaleuca oil (tea tree oil), Melissa oil, menthe arvensis oil, mountain savory oil, mugwort oil, mustard oil, myrrh oil, myrtle, neem oil, neroli oil, nutmeg oil, orange oil, oregano oil, orris oil, Palo Santo oil, parsley oil, patchouli oil, perilla essential oil, peppermint oil, petitgrain oil, pine oil, queen of the prairie oil, revensara oil, red cedar oil, roman chamomile oil, rose oil, rosehip oil, sage oil, sandalwood oil, sassafras oil, savory oil, schisandra oil, spearmint oil, spikenard oil, spruce oil, star anise oil, tangerine oil, tarragon oil, thyme oil, tsuga oil, turmeric oil, valerian oil, vetiver oil, western red cedar oil, wintergreen, yarrow oil, ylang-ylang oil, yucca oil, zedoary oil, one or more suitable carrier oils, one or more essential oils from any other plant, and any suitable combinations thereof.

While some embodiments of the described essential oil emulsification comprise a single essential oil, in other embodiments, the essential oil emulsification comprises any suitable number of essential oils, including, without limitation, a blend of 2, 3, 4, 5, 6, 7, 8, 9, 10, or more essential oils. In this regard, when the essential oil emulsification comprises more than one essential oil, the various oils can be present at any suitable ratio with respect to each other.

Due to the fact that some essential oils do not naturally dissolve in aqueous diluents, some embodiments of the present invention use an emulsifier. In this regard, the emulsifier can comprise any suitable chemical or chemicals that are capable of emulsifying one or more essentials oils so that the oils are miscible with, and can be diluted in, a diluent (discussed below). In this regard, some examples of suitable emulsifiers include, but are not limited to, polysorbate (e.g., polysorbate 20, polysorbate 40, polysorbate 60, polysorbate 80, etc.), sodium stearoyl lactylate, ceteareth 20, diacetyle tartaric (acid) ester of monoglyceride, lecithin, cetearyl alcohol, and/or any other suitable emulsifying agents. In some embodiments, however, the emulsifier comprises polysorbate 20.

The emulsifier can be mixed with the essential oil at any suitable ratio (by volume, molarity, etc.) that allows the essential oil or oils to be emulsified and diluted in a diluent (discussed below). In some embodiments, the emulsifying agent is mixed with the total amount of essential oil at a ratio (e.g., by volume) that is as high as a ratio selected from about 2:1 (emulsifying agent to essential oil), about 3:1, about 5:1, and about 10:1. In other embodiments, the emulsifying agent is mixed with the total amount of essential oil at a ratio that is as low as a ratio selected from about 1:10, about 1:5, about 1:3, and about 1:2. In still other embodiments, however, ratio between the total amount of the emulsifier and the total amount of the essential oil is any suitable combination or sub-range of the aforementioned ratios (e.g., between about 2:1 and about 1:2). Indeed, in some embodiments, the ratio of the total amount of the emulsifier to the total amount of the essential oil in the essential oil emulsification is between about 1.5:1 and about 1:1.5 (e.g., about 1:1).

The essential oil and emulsifier can be diluted in any suitable diluent, including, without limitation, water (e.g., de-ionized water, distilled water, tap water, etc.) and/or another aqueous solvent. Indeed, in some embodiments, the essential oil and/or emulsifier are diluted in distilled water.

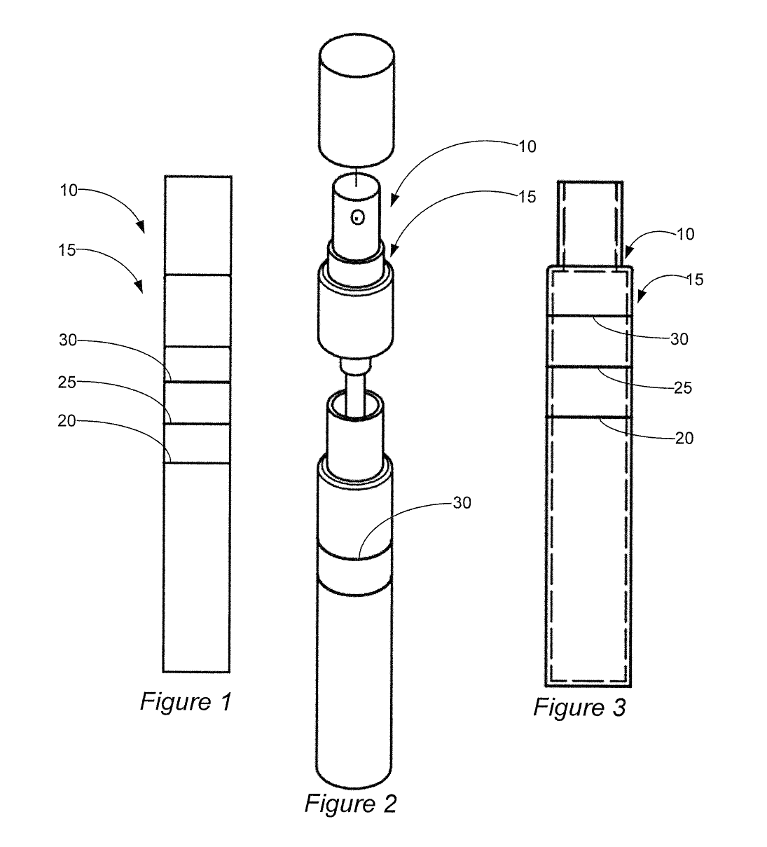

The essential oil and/or the emulsifier can be diluted at any suitable ratio (e.g., by volume, molarity, etc.) to the diluent that allows the emulsified essential oil to be at least partially dissolved or otherwise dispersed in the diluent. In some embodiments, the ratio (e.g., by volume) of the diluent (e.g., water) to the total amount of the essential oil (or the total amount of the emulsifier) is as high as a ratio selected from about 200:1, about 100:1, about 20:1, about 10:1, and about 7:1. In other embodiments, the ratio (e.g., by volume) of the diluent to the essential oil (or the emulsifier) is as low as a ratio selected from about 1:1, about 2:1, about 3:1, about 4:1, and about 5:1. In still other embodiments, the ratio of the diluent to the essential oil (or the emulsifier) is any suitable combination or sub-range of the aforementioned ratios (e.g., between about 5:1 and about 7:1, or between about 4:1 and about 8:1). Indeed, in some embodiments, the ratio of the diluent to the essential oil (or the emulsifier) is between about 5.5:1 and about 6.5:1 (or about 6:1). Thus, in some embodiments, while the essential oil and the emulsifier are present with respect to each other at a ratio of about 1:1, the diluent is present with respect to the essential oil and the emulsifier (individually) at a ratio of about 6:1. By way of non-limiting illustration, FIG. 1 shows that, in some embodiments in which the container 10 comprises about 8 ounces of liquid, the container is configured to hold about 1 fluid ounce of one or more essential oils A, about 1 fluid ounce of one or more emulsifiers B, and about 6 fluid ounces of a diluent C, such as water.

With respect to the container 10, the container can comprise any suitable vessel that is capable of holding the essential oil, emulsifier, and diluent. By way of example, the container can comprise a spray bottle, a vial, a jug, a canister, a can, a flask, a jar, and/or any other suitable receptacle. Indeed, in some embodiments, the container comprises a vessel, such as spray bottle, that is capable of spraying or otherwise diffusing an emulsified and diluted essential oil.

The container 10 can comprise any suitable mechanism that is capable of spraying or otherwise diffusing the essential oil emulsification. Some non-limiting examples of such mechanisms include a positive displacement pump, a negative displacement pump, a motorized pump, a piezoelectric pump, a pressurized canister, an aerosol canister, a nozzle (e.g., a spray nozzle, a nebulizer, an aspirator nozzle, an atomizer nozzle, a plain orifice nozzle, a shaped orifice nozzle, etc.), and/or another suitable mechanism of component that is capable of dispensing the essential oil emulsification as a mist, stream, cloud, spray, and/or in another manner that allows the emulsification to diffuse from the container. In some embodiments, however, the container comprises a manual pump (e.g., a pump that is actuated by depressing a nozzle, by pulling a trigger, etc.). By way of non-limiting illustration, FIGS. 2 and 3 each show a representative embodiment in which the container 10 comprises manual-pump spray bottle 15.

The container 10 can comprise any suitable material, including, without limitation, one or more plastics, metals, glasses, polymers, ceramics, and/or other suitable materials. In some embodiments, however, the container comprises a plastic bottle.

While the container can be any suitable size, in some embodiments, the container is portable. Indeed, in some instances, the container is capable of holding less than an amount selected from about 2 liters, about 1 liter, about 50 fluid ounces, about 20 fluid ounces, about 10 fluid ounces, about 2 fluid ounces, and about 1 fluid ounce. In one non-limiting example, the container is capable of holding about 8 fluid ounces.

In addition to the embodiments described above, the described systems and methods can be modified in any suitable manner. In one example, the essential oil emulsification is modified to include one or more additional ingredients. In this regard, some suitable examples of such additional ingredients include, but are not limited to, one or more perfumes, scents, antimicrobial agents, coloring agents, moisturizers, flavoring agents, preservatives, and/or other suitable ingredients.

In another example, instead of being provided with the essential oil, the container 10 is initially provided with the emulsifier and the diluent. Accordingly, a user (e.g., a store, end user, etc.) can create a customized dilution by adding one or more essential oils to the container (which contains the emulsifier and diluent) and then mixing its ingredients (e.g., via shaking or otherwise mixing the container's contents).

While the user can measure the amount of the essential oil that is added to the container 10 in any suitable manner, in some embodiments, the container comprises means for indicating the amount of essential oil (and/or emulsifier and/or diluent) that is to be included in the container. In this regards, the indicating means can comprise any suitable component or characteristic that indicates how much of any particular ingredient (e.g., the essential oil, emulsifier, and/or diluent) is to be included in the container.

In some embodiments, the container 10 comprises a translucent or transparent material that allows the user to see how much essential oil is being added to the container. In some embodiments, the container also comprises one or more markings (and/or other indicators) to indicate how much essential oil should be added to the container. In this regard, the container can be marked in any suitable manner that allows a user to understand how much of any ingredient (e.g., essential oil, emulsifier, and/or diluent) should be included in the container.

In one illustration, FIG. 3 shows a representative embodiment in which the container 10 comprises a marking 20 to indicate how much diluent is to be included in the container. Similarly, FIG. 3 shows an embodiment in which the container 10 includes a second marking 25 to indicate how much emulsifier should be included in the container. Additionally, FIG. 3 shows that, in some embodiments, the container 10 includes a third marking 30 to indicate how much essential oil should be included in the container. While FIG. 3 shows an embodiment, in which the container 10 comprises three markings, in other embodiments, the container can comprise any other suitable number of markings (e.g., 1, 2, 4, 5, 6, etc.). For instance, in some embodiments in which the container 10 is provided with the emulsifier and the diluent, the container comprises one or more markings that simply indicate how much essential oil is to be added to the container.

In some embodiments, instead of (or in addition to markings), the container 10 (as shown in FIG. 2) is sized such that once it is filled with a proper amount of diluent and emulsifier, the container will not allow more than a suitable amount of essential oil to be added to the container.

In still another example, the container 10 comprises a space that allows a user to write what oil or oils (other ingredients) were added to the solution for identification purposes.

In addition to the aforementioned characteristics, the described systems and methods may include one or more beneficial characteristics. In one example, the described systems allow a user to dilute and/or diffuse an essential oil for its aromatic benefits. For instance, some embodiments of the described invention provide a portable air freshener. In another example, some embodiments of the described systems and methods allow a user to easily carry the emulsified and diluted oil (e.g., in a pocket, purse, etc.). In still another example, as some embodiments of the described invention do not require an electrical power source (e.g., an electrical socket or battery), such embodiments can be free from shortcomings associated with such power sources. In still another example, the described systems and methods can allow users (e.g., representatives of essential oil companies) interested in selling essential oils to have a simple, convenient, and cost-effective way to dilute and share the aroma of virtually any essential oil. For instance, instead of giving potential customers a sample of a pure essential oil, which may be relatively costly, the user could spray a diluted amount of the essential oil into the air or onto substrate (e.g., a small card) for potential clients to take home with them. In still another example, because some embodiments of the invention are portable, the described systems and methods can be used to spray essential oil in a car, office, or virtually any other suitable location. Accordingly, such embodiments can perform one or more beneficial functions, including, without limitation, dispensing one or more essential oils as an air freshener, as a disinfectant, etc.

Thus, as discussed herein, embodiments of the present invention embrace essential oils. In particular, the present invention relates to systems and methods for diluting one or more essential oils and diffusing such oils from a portable dispenser.

The present invention may be embodied in other specific forms without departing from its spirit or essential characteristics. The described embodiments and examples are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. An essential oil emulsion comprising:
an essential oil;
an emulsifier; and
an aqueous diluent,
wherein a ratio of the essential oil to the emulsifier is between about 2:1 and about 1:2, and
wherein a ratio of the aqueous diluent to the essential oil is between about 5:1 and about 8:1.

2. The emulsion of claim 1, wherein the emulsion is disposed in a container having a manual pump that is configured to diffuse the emulsion from the container.

3. The emulsion of claim 1, wherein the emulsifier comprises polysorbate.

4. The emulsion of claim 1, wherein the ratio of the essential oil to emulsifier is between about 1.5:1 and about 1:1.5.

5. The emulsion of claim 1, wherein the ratio of the essential oil to emulsifier is about 1:1.

6. The emulsion of claim 1, wherein the ratio of the aqueous diluent to the essential oil is between about 5.5:1 and about 6.5:1.

7. A vessel comprising an essential oil emulsion, the vessel comprising:
a container configured to diffuse the essential oil emulsion from the container; and
the essential oil emulsion, wherein the emulsion comprises:
an essential oil;
an emulsifier; and
an aqueous diluent,
wherein a ratio of the essential oil to emulsifier is between about 2:1 and about 1:2, and
wherein a ratio of the aqueous diluent to the essential oil is between about 5:1 and about 8:1.

8. The vessel of claim 7, wherein the container comprises a manual pump configured to expel a portion of the emulsion from the container.

9. The vessel of claim 7, wherein the emulsifier comprises polysorbate.

10. The vessel of claim 7, wherein the ratio of the essential oil to emulsifier is between about 1.5:1 and about 1:1.5.

11. The vessel of claim 10, wherein the ratio of the aqueous diluent to the essential oil is between about 5.5:1 and about 6.5:1.

12. The vessel of claim 7, wherein the ratio of the essential oil to emulsifier is about 1:1, and wherein the ratio of the aqueous diluent to the essential oil is about 1:6.

13. An essential oil container comprising:
a container comprising:
an aqueous diluent; and
an emulsifier capable of emulsifying an essential oil in the aqueous diluent,
wherein a ratio of the emulsifier to the aqueous diluent in the container is between about 1:4 and about 1:8, and
wherein the container comprises sufficient space to receive a volume of an essential oil that provides a ratio between the volume of the essential oil and a volume of the emulsifier of between about 2:1 and about 1:2.

14. The container of claim 13, wherein the container comprises means for indicating the amount of the essential oil that is to be added to the container.

15. The container of claim 13, wherein when the container is initially provided with the aqueous diluent and the emulsifier, the container has enough room to receive the essential oil at a ratio of no more than 2 volumes of the essential oil to 1 volume of the emulsifier in the container.

16. The container of claim 13, wherein the emulsifier comprises polysorbate.

17. The container of claim 13, wherein the container indicates an amount of the aqueous diluent and an amount of the emulsifier that are to be included in the container.

18. The container of claim 17, wherein the container further indicates an amount of the essential oil that is to be included in the container.

19. The container of claim 13, wherein the ratio of the aqueous diluent to the emulsifier in the container is between about 5.5:1 and about 6.5:1.

20. The container of claim 13, wherein the container comprises an aerosol can.
